(19) United States
(12) Patent Application Publication
(10) Pub. No.: US 2007/0277836 A1
(43) Pub. Date: Dec. 6, 2007

(54) Apparatuses, Systems and Methods for Determining Compliant Use of an Oral Appliance

(76) Inventor: William H. Longley, Atlanta, GA (US)

Correspondence Address: Robert A. Blaha;SMITH FROHWEIN TEMPEL GREENLEE BLAHA LLC, Suite 700, Two Ravinia Drive, Atlanta, GA, 30346, US

(21) Appl. No.: 11/754,894

(22) Filed: May 29, 2007

Related U.S. Application Data
(63) Continuation-in-part of application No. 11/422,279, filed on Jun. 5, 2006.
Application No. 11/422,279 is a continuation-in-part of application No. 11/531,620, filed on Sep. 13, 2006.

(51) Int. Cl. A61F 5/56

(52) U.S. Cl. 128/848

(57) ABSTRACT

An information system determines compliant or non-compliant use of an oral appliance over time. The system includes a communication device and a data analyzer. The communication device forwards commands to, and receives information from, an oral appliance. The oral appliance records at least one measure indicative of presence of the oral appliance in an oral cavity. The communication device communicates with the oral appliance when the two are within operational range of each other. The communication device forwards information to the data analyzer. In turn, the data analyzer determines whether a user's use of the oral appliance is compliant with a schedule. The data analyzer generates one or more reports that can be accessed, communicated and reviewed by those with appropriate authorization credentials.

### CROSS REFERENCE TO RELATED APPLICATIONS

This application is a continuation-in-part and claims priority from U.S. patent application Ser. No. 11/422,279, entitled “Apparatuses, Systems and Methods for Confirming Use of an Apparatus,” filed on Jun. 5, 2006, and is a continuation-in-part and claims priority from U.S. patent application Ser. No. 11/531,620, entitled “Apparatuses, Systems and Methods for Monitoring Conditions in an Oral Cavity,” filed on Sep. 13, 2006, the entire disclosures of which are incorporated herein by reference.

### BACKGROUND

A number of oral appliances have been developed in association with various therapies related to the treatment of sleep apnea, snoring, bruxism, orthodontia, eating disorders and other medical and dental issues.

Difficulty in breathing while sleeping often manifests itself as snoring or the more serious obstructive sleep apnea. Snoring is a condition affecting approximately forty percent (40%) of the adult population, while sleep apnea is believed to affect at least twenty (20) million people.

Bruxism consists of a static and/or dynamic contact between the dentition of the mandible and maxilla. Bruxism is commonly referred to as “tooth grinding,” which results in the physical destruction of tooth enamel directly leading to tooth decay as well as more serious problems. Bruxism affects nearly fifteen percent (15%) of the population and in advanced stages bruxism results in the abnormal and excessive grinding or clenching of teeth while an individual is asleep. Bruxism is medically classified as parasomnia or sleep disorder.

In turn, temporomandibular joint syndrome (or dysfunction) (“TMJ”) is a disorder effecting the joint between the lower jaw and the skull. The temporomandibular joint lies between the temporal bone of the skull and the mandible of the jaw, and allows the jaw to open and close. The joint is formed by a condyle on the mandible, which hinges and glides in and out of the depression in the temporal bone.

TMJ can be caused by bruxism, malocclusion, trauma, and arthritis. There is also an indication that a posterior or backward displacement of the condyle of the jaw significantly contributes to TMJ pain. TMJ has been associated with a wide variety of physical aliments, including migraine headaches. TMJ related headaches can become so severe as to cause nausea and blurred vision. Most people afflicted with TMJ suffer from a myofascial pain-dysfunction syndrome primarily as a muscle problem related to dental/skeletal relationships and tensional factors. The effects can range from mild to severe, including pain in the joint area that can extend to the shoulders, back, neck, and sinuses.

U.S. Pat. No. 6,769,910 discloses a non-surgical oral appliance for improving breathing, and abating or completely alleviating snoring sounds, TMJ and bruxism while sleeping. In one embodiment, the user is professionally fitted for the appliance such that the appliance positions the mandible in an open, protrusive, predetermined position such that the oral airway permits the enhanced passage of air.

U.S. Pat. No. 4,609,349 discloses an orthodontic device that includes a tubular member secured to the surface of an oral cavity portion, which may serve as a tensioning member or a mount for a wire spring-like tensioning member. The member is positioned in such a manner as to be in alignment with the tooth to be straightened. A number of bends in the tubular member and the tensioning member allow for the exertion of a force over a relatively long distance. The device may be used on either the upper or lower teeth. The orthodontic device is representative of one type of many different orthodontic devices that are inserted and removable by a patient. To achieve the desired therapeutic effect, such devices must be used for a significant portion of each day. It has been reported that estimates of compliance by patients, parents and their orthodontic professionals are mostly inaccurate.

Oral appliances have been developed and prescribed in association with conditions in addition to the above-described oral, respiratory, dental and developmental disorders. According to the American Obesity Association (AOA), obesity is a disease that affects nearly one-third of the adult American population. The number of overweight and obese Americans has continued to increase since 1960—a trend that is not slowing down. Research conducted by the AOA indicates that 64.5 percent of adult Americans (about 127 million) are currently categorized as being overweight or obese.

Each year, obesity causes at least 300,000 excess deaths in the United States alone, and healthcare costs of American adults with obesity amount to approximately $100 billion.

Individuals with obesity are at risk of developing one or more serious medical conditions, which can cause poor health and premature death. For instance, research suggests that obesity is statistically linked to adverse medical conditions, such as diabetes, coronary heart disease, high blood pressure, osteoarthritis, hypertension, and cardiovascular disease to a name a few of the more severe conditions. The problems associated with obesity and other less severe weight issues are not limited to medical conditions. Obese and overweight people may also suffer from a number of other problems (e.g. lifestyle problems, psychological conditions, social problems, etc.).

Currently, there are a number of weight loss methods and devices for enabling people to attempt to manage their weight problems. The most prevalent methods include dieting, exercising, medications, body wrapping, surgical procedures, etc. Other weight management solutions involve mechanical devices and dental appliances. One such device is positioned in the mouth to stimulate salivation and swallowing, such as disclosed in U.S. Pat. Nos. 3,224,442 and 5,052,410.

U.S. Pat. No. 5,924,422 discloses a removable, retainer-like device that is molded to fit the contours and configuration of the user's mouth. The upper surface of the retainer-like device is configured to fit the roof of the user's mouth, while the lower surface is configured to replicate the roof, or palate, of the user's mouth. The retainer-like device is adapted to be easily and quickly put into use, without any special training, by positioning it in the mouth with the upper surface against the palate, and pushing the retainer between the corresponding teeth. When worn, the device effectively lowers the roof of the user's mouth to reduce the overall volume of the oral cavity. The device is intended to be easily inserted and removed by the user.

Regardless of the malady that a particular oral appliance is attempting to treat, the oral appliance, if it is to be effective, should be used in accordance with a prescribed usage schedule to attain the desired therapeutic benefits. Accordingly, the dental and medical professions, medical and dental insurers, parents and others interested in a user's compliance with a prescribed usage schedule for a respective device, recognize the paramount importance of developing systems and methods for ensuring a user's compliant use of an oral appliance.

### SUMMARY

An embodiment of an information system for determining compliant use of an oral appliance comprises a communication device and a data analyzer. An oral appliance is configured to record periodically at least one datum indicative of the presence of the oral appliance in an oral cavity. The communication device is configured to communicate with the oral appliance when the oral appliance and the communication device are within range of one another. The data analyzer receives the information from the oral appliance via the communication device. The data analyzer then determines whether a user's use of the oral appliance is compliant with a schedule.

An embodiment of a method for confirming compliant use of an oral appliance comprises the steps of providing an oral appliance to a user, the oral appliance configured to record information indicative of placement of the oral appliance in an oral cavity over time, using a communication device to direct the oral appliance to transfer the information, and analyzing the information to determine whether use of the oral appliance is compliant with a schedule.

Other apparatuses, systems, methods, features and advantages will be or will become apparent to one with skill in the art upon examination of the following figures and detailed description. All such additional apparatuses, systems, methods, features and advantages are defined and protected by the accompanying claims.

### BRIEF DESCRIPTION OF THE FIGURES.

The apparatuses, systems and methods for determining compliant use of an oral appliance can be better understood with reference to the following figures. The components within the figures are not necessarily to scale; emphasis instead is placed upon clearly illustrating the principles involved. Moreover, in the figures, like reference numbers designate corresponding parts throughout the different views.

FIG. 1 is a schematic diagram illustrating components of an information system for determining compliant use of an oral appliance.

FIGS. 2A and 2B are schematic diagrams of example embodiments of oral appliances that can be integrated with the information system of FIG. 1.

FIG. 3 is a functional block diagram illustrating an embodiment of the oral appliance of FIG. 1.

FIG. 4 is a schematic diagram illustrating an embodiment of a memory device within the recorder of FIG. 3.

FIG. 5 is a schematic diagram illustrating an embodiment of the sensor assembly of FIG.3.

FIG. 6 is a functional block diagram illustrating an embodiment of the communication device of FIG. 1.

FIG. 7 is a functional block diagram illustrating an embodiment of the data analyzer of FIG. 1.

FIGS. 8A-8C are schematic diagrams illustrating alternative embodiments of a report generated by the data analyzer of FIG. 1.

FIG. 9 is a flow diagram illustrating an embodiment of a method for determining compliant use of an oral appliance.

FIG. 10 is a flow diagram illustrating an embodiment of an alternative method for determining compliant use of an oral appliance.

FIG. 11 is a flow diagram illustrating an embodiment of another method for determining compliant use of an oral appliance.

### DETAILED DESCRIPTION

An oral appliance is constructed with a housing or chamber that substantially encloses one or more sensors, a recorder, and a transceiver. The oral appliance responds to commands received via from a communication device integrated with an information processing system. The communication device includes a transceiver configured to send commands and receive information from the oral appliance. The oral appliance is reset or otherwise configured to record periodically a measurement or measurements for a desired length of time. The oral appliance is prescribed or otherwise provided to a user of the oral appliance. Preferably, before the desired length of time has expired, the user transfers recorded data to via the communication device to a data analyzer. This can be accomplished by returning the oral appliance to the provider of the device or providing the user with a suitably configured communication device that receives recorded data from the oral appliance and forwards the data to the provider to the provider of the device. After it is initialized, the oral appliance continues to record measurements. When communicatively coupled to a suitably configured communication device, the oral appliance receives commands that direct the appliance to transmit the recorded measurements. The transmitted measurements can be used to determine whether the user of the oral appliance has complied with a recommended usage schedule.

A data analyzer identifies, collects and organizes the information from the oral appliance via the communication device. The data analyzer is part of an information processing system that determines and reports compliant or non-compliant use of the oral appliance under the direction of the provider of the oral appliance. In described embodiments, the provider is an orthodontist who has constructed the oral appliance and instructed a patient regarding its proper use. The information processing system not only collects and organizes the information to determine compliant use, but provides an interface to the orthodontist, the patients and their parents or caregivers to communicate with each other and view one or more reports that compare the patient's use with the prescribed use and compare the patient's use with other patients using similar oral appliances.

In one embodiment, the oral appliance is configured to encrypt data before the data is transferred to the communication device. Data encryption can take place before the data is stored in the oral appliance or as part of the process of transferring the information to the communication device. For example, once the oral appliance is placed within range of the communication device and a communication link is established, the communication device creates a symmetric key and forwards the key to the oral appliance via the link. The key can be transferred using public-key encryption. The oral appliance can then communicate stored data by encrypting the data using symmetric-key encryption. Once the session is finished, both the communication device and the oral appliance can be programmed to discard the symmetric key used for that session. Any additional or subsequent sessions will use a new symmetric key.

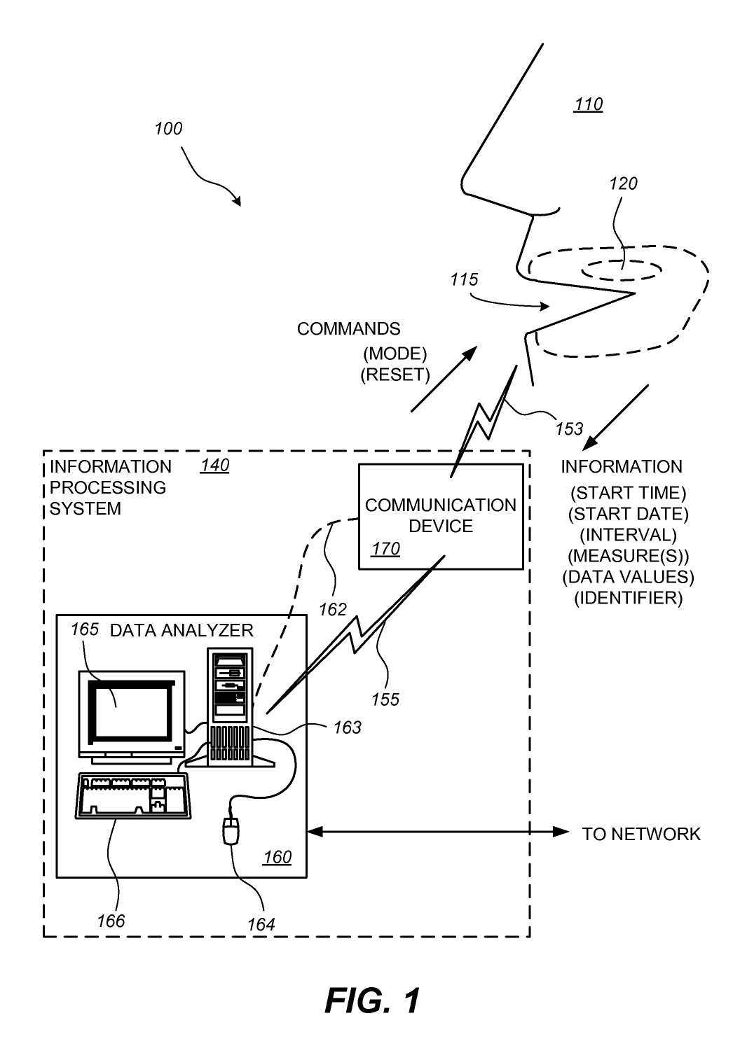

Having generally described the systems and methods for determining compliant use of an oral appliance, various additional embodiments will be described with respect to FIGS. 1-11. By way of example, FIG. 1 is a side view of the head of a user 110 that is using an oral appliance 120. By way of example, FIG. 1 is a schematic diagram illustrating components of an information processing system 140 that receives information from oral appliance 120, determines intervals of compliant use and communicates various notices and reports to interested parties in accordance with system parameters. Information processing system 140 includes communication device 170 and data analyzer 160. As illustrated in FIG. 1, communication device 170 transmits commands and receives information from oral appliance 120. The illustration shows that wireless commands can be transmitted to and information can be received from oral appliance 120 while it is positioned in an oral cavity 115. This is not the only arrangement that supports two-way communications between communication device 170 and oral appliance 120. For example, two-way communications can be established via wireless link 153 when the oral appliance is removed from the oral cavity and is located within several feet of communication device 170.

In the illustrated embodiment, data analyzer 160 is a workstation that includes an enclosure 163 for housing a power supply, central-processing unit, input/output interface controllers as well as fixed and removable data-storage devices. Enclosure 163 is coupled to input/output devices such as mouse 164, display 165 and keyboard 166. Enclosure 163 is coupled to a network via a suitably configured network interface device. The network can be a local-area network, a wide-area network or a combination network. Furthermore, the network interface device can use a wired or a wireless medium to communicate information to network coupled devices.

Data analyzer 160 is further coupled to communication device 170 via wired link 162 or wireless link 155. Communication device 170 is coupled to apparatus 120 via wireless link 153. Wired link 162 can be implemented via any of the packet-based communication protocols commonly known as Ethernet or via one or more standard or proprietary communication protocols for operating a parallel or serial data bus. Wired link 162 may also include circuits for the distribution of power. Wireless link 153 and wireless link 155 can be implemented via any of the short-range radio frequency communication protocols (e.g., 802.11, 802.15.1) or any of the infra-red spectrum based communication protocols (e.g., IrDA).

While wired link 162, wireless link 153 and wireless link 155 are shown together in the illustrated embodiment, one, two or all of the links may be inactive at any given time. For example, most of the time user 110 will not be proximally located to communication device 170. Under these circumstances, a communication session between oral appliance 120 and communication device 170 will not be possible because of the limited effective range of the transceivers in the oral appliance 120 and communication device 170. When communication device 170 is configured with one or more transceivers that use the infra-red spectrum, communication sessions are possible when user 110 is not only proximally located to communication device 170, but the respective transceivers in oral appliance 120 and communication device 170 must be arranged such that the emitter in one is aligned and not obstructed from the infra-red sensitive sensor in the other. When communication device 170 is configured with a self-contained power supply (e.g., a battery) and a memory capacity that exceeds that necessary to store the recorded information in oral appliance 120, only one of wireless link 153 or wireless link 155 may be active at any given time.

Data analyzer 160 is configured with software or firmware to transfer and store information received from oral appliance 120 via communication device 170. Data analyzer 160 is further configured with software suited to organize, display, analyze or otherwise interpret the recorded information to determine when the oral appliance is being used in accordance with a prescribed schedule.

As described in greater detail below, data analyzer 160 is arranged with a database, authentication logic, analyzer logic and report logic. The database collects and organizes information from multiple communication sessions established with the communication device 170 to receive information from oral appliance 120 at designated times. Authentication logic verifies that various users of the system are only exposed to patient information and/or configuration information in accordance with access credentials associated with each user of the system. Authentication logic confirms or authenticates operators of computing devices that attempt to establish a communication session with data analyzer 160.

A web server integrated with data analyzer 160 enables an orthodontist or other professional, as well as patients and caregivers access to the information. In addition to providing access to the information in its most basic forms, analyzer logic and report logic work together to determine and log when the information indicates that the user 110 has used the oral appliance 120. Report logic can be configured by the orthodontist or other professional to generate notices of non-compliant use via one or more of email, voicemail, text messages or a patient/caregiver accessible web page. Reports can be generated and forwarded in accordance with a plan or can be generated and supplied in near real time in accordance with a request communicated by an operator of a computing device coupled to data analyzer 160 via one or more networks. Planned or scheduled communications can be sent as email attachments over a data network, via an automated voicemail system over the public-switched network or as text messages over the public-switched network and a cellular network. Operators of computing devices that access data analyzer 160 via a uniform resource locator are prompted to enter a user name and passcode before being routed to a report generation interface page.

The illustrated elements of information processing system 140 can be collocated or removed from one another. In one embodiment, elements of information processing system 140 can be located in an orthodontist's office. Alternatively, one or both of the devices (i.e., the communication device 170 and the data analyzer 160) will be removed from the orthodontist's office. In these embodiments, the communication device 170 can be provided to the patient or the patient's parents to enable periodic data transfers or communication device 170 can be located in the orthodontist's office where it will be available for data transfers when the patient is present. Similarly, data analyzer 160 can be hosted on a network coupled server that is accessible to both the orthodontist and the communication device 170 via one or more wireless or wired networks. When this is the case, the network coupled server will control access to patient data by granting access to patients and/or their parents and the orthodontist in accordance with one or more rules.

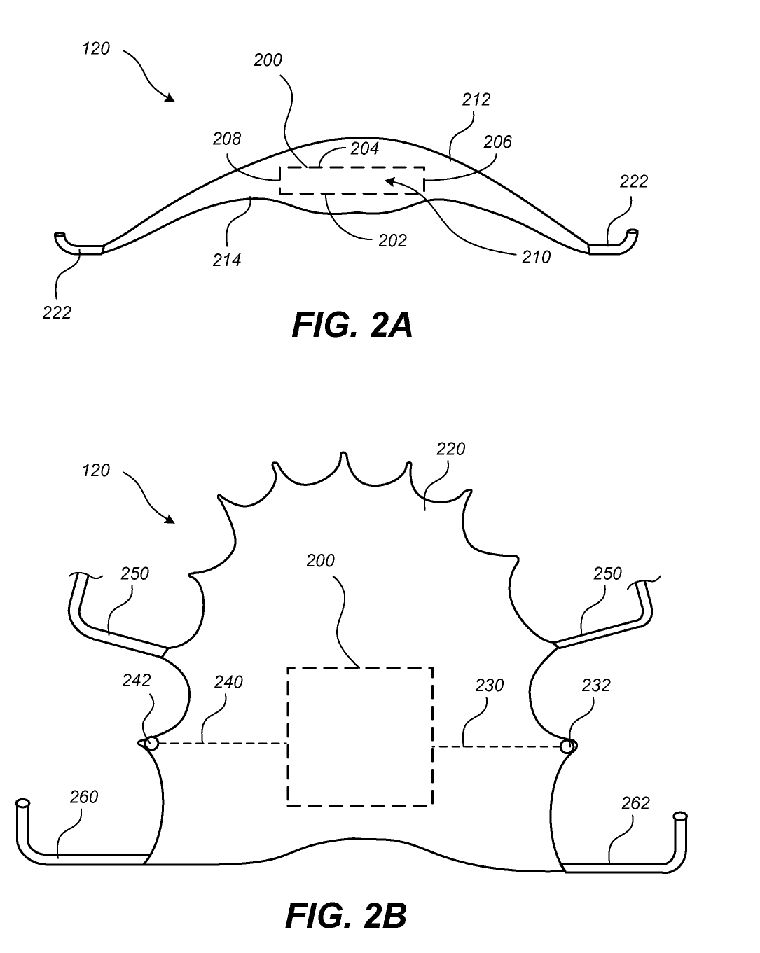

FIG. 2A is a schematic diagram illustrating an embodiment of the oral appliance 120 of FIG. 1. In the illustrated embodiment, oral appliance 120 is a device for lowering the upper palate of the user's oral cavity to reduce the volume that can be contained in the oral cavity. The oral appliance 120 can be fitted by a user of the appliance by arranging probes 222 on either side of the appliance to fit in the user's upper dentition. The oral appliance 120 includes a housing 200 (shown with hidden lines) that forms an internal cavity 210 within a dental acrylic. As will be explained in greater detail below, housing 200 protects elements configured to periodically sense and record one or more environmental conditions surrounding the oral appliance 120 to identify to a reviewer of the recorded information the usage habits of the user 110 of oral appliance 120. In the embodiment illustrated in FIG. 2A, oral appliance 120 is shown upside down. In use, upper surface 212 extends into the oral cavity in a direction from the roof of oral cavity towards the upper surface of the patient's tongue. Lower surface 214 is shaped such that when the oral appliance is placed in the oral cavity a void remains between the roof of the patient's mouth and the oral appliance 120. The void provides enough separation so that food can be rinsed or otherwise removed from above the oral appliance 120. Housing 200 comprises a lower surface 202, an opposing upper surface 204, a right-side surface 206 and an opposing left-side surface 208. It should be understood that alternative arrangements are possible including those where the dental acrylic conformally coats portions of one or more sensors and entirely coats the power source, recorder and transceiver.

FIG. 2B is a top view illustrating an alternative embodiment of the oral appliance 120 of FIG. 1. In the illustrated embodiment, oral appliance 120 is a device for retaining or holding the position of a patient's upper teeth. The oral appliance includes body 220, which has been constructed from a mold of the patient's oral cavity. The oral appliance 120 can be fitted by a user of the oral appliance by arranging probe 260 and probe 262 on either side of the oral appliance to fit in the user's upper dentition. Tensioning member 250 extends from opposing sides of body 220 and provides sufficient force to hold or retain each of the upper teeth in a desired configuration. Body 220 includes a housing 200 (shown with hidden lines) that forms an internal cavity within a dental acrylic used to form body 220. As will be explained in greater detail below, housing 200 protects elements configured to periodically sense and record one or more environmental conditions surrounding the oral appliance 120 to identify to a reviewer of the recorded information the usage habits of the user 110 of oral appliance 120.

In use, a pressure sensitive switch 232, arranged along the right side of body 220, engages or contacts the roof of the patient's mouth near the intersection of two molars. Pressure sensitive switch 232 is coupled to a sensor assembly (not shown) arranged in housing 200. Similarly, pressure sensitive switch 242, arranged along the left side of body 220, engages or contacts the roof of the patient's mouth near the intersection of two molars. Pressure sensitive switch 242 is coupled to a sensor assembly (not shown) arranged in housing 200. Pressure sensitive switches 232, 242 provide an indication that the patient has arranged the oral appliance 120 in the intended position in the oral cavity 115. Additional sensors (not shown) will provide information regarding other conditions expected to be encountered in the oral cavity 115.

The oral appliances illustrated in FIGS. 2A and 2B are by way of example only. Other orthodontic, dental or therapeutic aids can be arranged with a suitably configured sensor array, recorder and transceiver to communicate with the information processing system. Such other devices include but are not limited to mouth guards to prevent bruxism, snoring or sleep apnea as well as devices used for the retention or realignment of teeth.

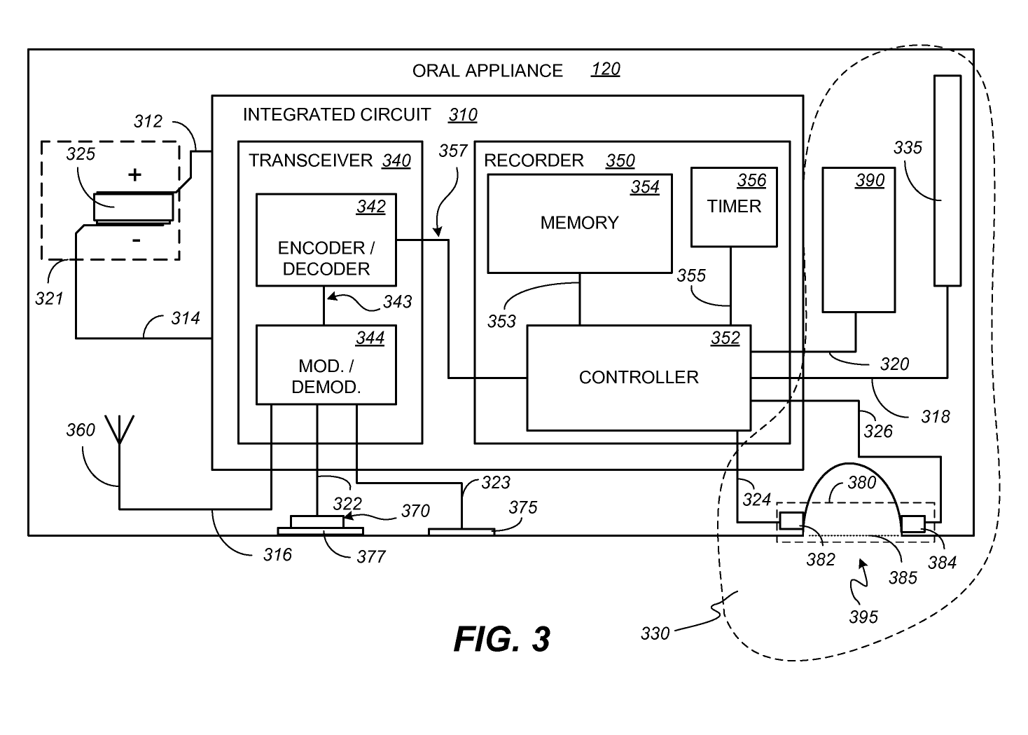

FIG. 3 is a schematic diagram illustrating the arrangement of a power source 321 coupled to a sensor array 330, a recorder 350 and a transceiver 340 on an integrated circuit 310 within oral appliance 120. In the illustrated embodiment, oral appliance 120 entirely encompasses integrated circuit 310, power source 321, sensor array 330, and antenna 360. In alternative embodiments (not shown), sensor array 330 or other sensors such as sensor 380 may extend to or through a surface of oral appliance 120 such that a portion of a sensor is exposed to the user's oral cavity.

As illustrated in FIG. 3, power source 321 is coupled and provides power to integrated circuit 310. Specifically, a positive terminal of power source 321 is electrically connected to a power input pin via conductor 312 and a negative terminal of power source 321 is electrically connected to a ground input pin via conductor 314.

Integrated circuit 310 includes recorder 350 and transceiver 340. Sensor array 330 is coupled to recorder 350 via link 318. Sensor 380 is coupled to recorder 350 via link 324 and link 326. Sensor 380 encloses saliva and other liquids, which translate through membrane 385 from the user's oral cavity. Link 324 is coupled to measurement electrode 382. Link 326 is coupled to reference electrode 384. Transceiver 340 is coupled to recorder 350 via link 357. Transceiver 340 is coupled to antenna 360 via link 316. Transceiver 340 is further coupled to infrared emitter/sensor 375 via link 323. Transceiver 340 is also coupled to connector 370 via link 322. In the illustrated embodiment, connector 370 is protected from the user's oral cavity via cover 377. Cover 377 is arranged so that it can be removed when a technician desires to communicatively couple transceiver 340 via a wired connection to external communication devices such as communication device 170 and replaced when oral appliance 120 is returned to the user.

Sensor array 330 is responsive to temperature, humidity, contact and pH. Sensor 335 is responsive to temperature within a range of expected values. Allowances for reasonable short term variation due to ingested food or beverages can be made at the discretion of the orthodontist. Sensor 380 is responsive to humidity and pH present in the patient's oral cavity 115. It should be understood that alternative arrangements of sensor 380 (not shown) may be devised to detect the presence of other items in the patient's oral cavity as may be desired. Sensor 390 is sensitive to contact. In this regard, sensor 390 may be arranged on an external surface of oral appliance 120 that is expected to contact upper or lower teeth or other portions of a patient's oral cavity 115.

Recorder 350 includes controller 352, memory 354 and timer 35655. Controller 352 is coupled to memory 354 via bus 353. Controller 352 is further coupled to timer 356 via link 355 and transceiver 340 via link 357.

Transceiver 340 includes encoder/decoder 342 and modulator/demodulator 344. Encoder/decoder 342 is coupled to modulator/demodulator 344 via bus 343.

Encoder/decoder 342 can be configured to receive a symmetric key from communication device 170 and as previously described use the symmetric key to encrypt data stored in memory 354 before transmitting the stored data to communication device 170.

In operation, a radio-frequency signal containing one or more commands from a suitably configured communication device is received via antenna 360 and a tuner (not shown). The received signal is forwarded to modulator/demodulator 344 via link 316.

When the oral appliance 120 is coupled via a wired connection to a communication device (not shown), commands can be forwarded to modulator/demodulator 344 via connector 370 and link 322. Alternatively, when oral appliance is coupled via an infrared communication link, an infrared signal may be received via infrared sensor 375 and forwarded to modulator/demodulator 344 via link 323.

Modulator/demodulator 344 detects and separates information from the received signal. The information is forwarded to encoder/decoder 342 via bus 343. Encoder/decoder 342 converts the received information to a format compatible with controller 352. Controller 352 responds in accordance with the one or more received commands. For example, an identifier set command includes a unique identifier that can be stored in memory 354, a start time reset command includes information responsive to a time or a time and date, an interval set command includes information that defines a time interval between measurements, a transmit command instructs controller 352 to read and communicate each of the recorded measurements, a clear command directs controller 352 to remove recorded measurements from memory 354. Additional commands including commands that define a mode of operation can be communicated to oral appliance 120. A suitably configured communication device may send multiple commands when oral appliance 120 is within range (in a wireless data transfer mode) or directly coupled via connector 370 and link 322 (in a wired data transfer mode). As explained above, antenna 360 and connector 370 as well as link 316 and link 322 could be replaced by infrared sensor 375 and link 323 to communicate wirelessly with external computing devices.

During a session, which is defined as the time between a start time reset command and a transmit command, controller 352 in accordance with periodic signals received via link 355 from timer 356 latches at least one current or a voltage provided by sensor array 330. In some operational modes, controller 352 directs oral appliance 120 to record measures from different sensors to reduce the probability of the data analyzer 160 reporting a false positive use of the oral appliance when the user places the oral device in an environment that resembles the environment of an oral cavity.

In some embodiments, controller 352 is configured with an analog to digital converter, which generates a digital representation of the analog output from sensor array 330. In these embodiments controller 352 simply forwards the latched and digitized measurement into the next available location within memory 354. In other embodiments, memory 354 is configured with calibration information, which is used to convert the recorded measurement via a scale factor. When the sensor array 330 in these other embodiments is responsive to temperature, the scale factor may result in memory 354 storing data values in degrees Fahrenheit or degrees Celsius. When the sensor 330 in these other embodiments is responsive to humidity, the scale may be a percentage from 0% (an environment devoid of water vapor) to 100% (an environment completely saturated with water vapor). Sensor 380, as described above, may produce relative voltages, the polarity and magnitude of which are indicative of the pH concentration in the oral cavity 115. Regardless of the nature of the various sensors in sensor array 330, data values are forwarded and stored in the next available location within memory 354 in accordance with a signal from timer 356.

Memory 354 includes adequate storage locations to store measurements for an extended session. When the user 110 of oral appliance 120 fails to place the oral appliance 120 within signal range of a suitably configured communication device and controller 352 has forwarded a measurement to each available memory location within memory 354, subsequent measurements will be forwarded to and will overwrite measurement information in the same sequence as previous measurements were stored in memory 354. It should be understood that when oral appliance 120 is within range of communication device 170 or directly coupled to communication device 170, measurements can be sent from oral appliance 120 to communication device 170.

In turn, communication device 170 can buffer the received information until such time that a communication session is established with data analyzer 160. Alternatively, when communication device 170 is directly coupled to data analyzer via link 162 (FIG. 1), communication device 170 may temporarily buffer and transfer information from oral appliance 120 to data analyzer 160 in accordance with wireless and wired communication protocols.

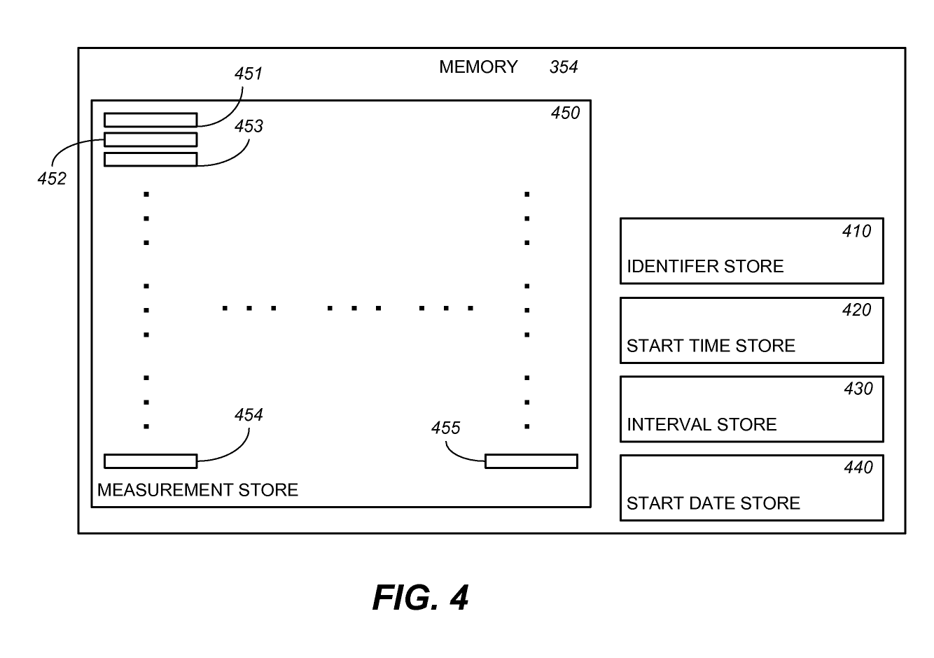

FIG. 4 is a schematic diagram illustrating an embodiment of storage locations within the memory 354 of FIG. 3. As shown in FIG. 4, memory 354 includes identifier store 410, start time store 420, interval store 430, start date store 440 and measurement store 450. Identifier store 410 is a designated memory location for a unique identifier for oral appliance 120. Start time store 420 is a designated memory location for a start time. Interval store 430 is a designated memory location for an interval or duration of time that controller 352 will wait between recording and storing an environmental parameter as determined by sensor array 330. Start date store 440 is a designated memory location for a start date. As further indicated in FIG. 4, measurement store 450 includes many designated memory locations, such as location 451, location 452, location 453 through to location 454 in a leftmost column of locations, through to location 455, the last location in a rightmost column of measurement store 450. Each of location 451 through location 455 may include a data values from a particular sensor in oral appliance 120 or multiple data values from two or more respective sensors in accordance with a present operational mode. It should be understood that each of the individual measurement locations could be arranged in other sequences separate and distinct from the sequence shown in FIG. 4.

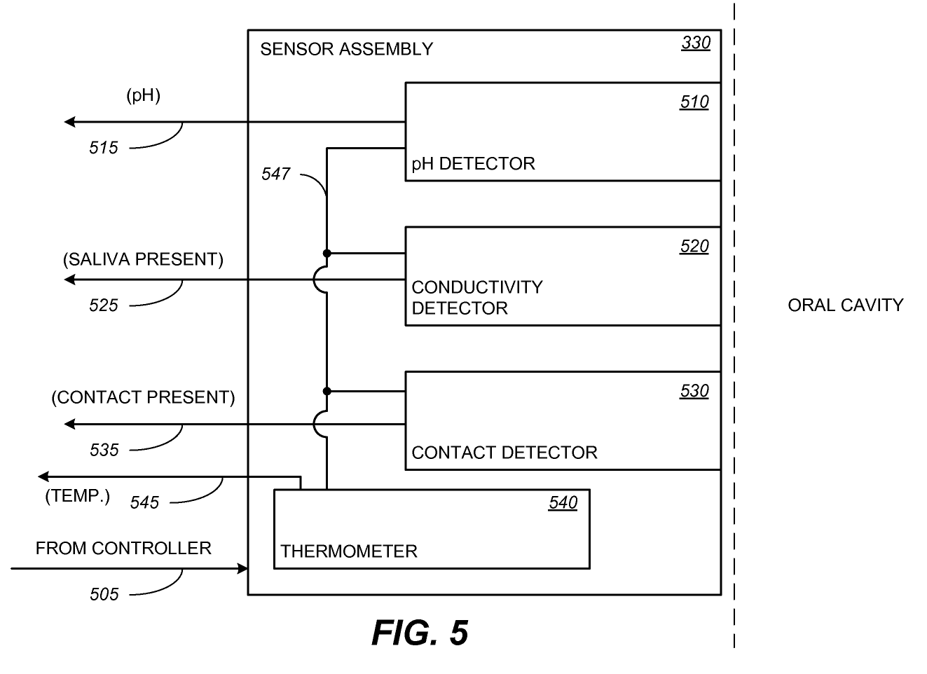

FIG. 5 is a schematic diagram illustrating an embodiment of the sensor assembly 330 of FIG. 3. Sensor assembly 330 receives configuration information and power from controller 352 (FIG. 3) via connection 505. Sensor assembly 330 provides a host of information signals responsive to the presence of one or more conditions in the patient's oral cavity. Sensor assembly 330 includes various sensors configured to detect temperature, contact, saliva/humidity and the concentration of hydrogen ions (pH) in a patient's oral cavity. In the example embodiment, sensor assembly includes thermometer 540, pH detector 510, conductivity (humidity) detector 520, and contact detector 530.

Thermometer 540 generates a signal responsive to the temperature within the host's oral cavity. Thermometer provided information can be used to determine when the host has inserted the oral appliance 120 in the oral cavity 115. Thermometer 540 is coupled to pH detector 510, conductivity detector 520, and contact detector 530 via bus 547. Thermometer 540 is also coupled to controller 352 (FIG. 3) via bus 545. The signal from thermometer 540 can be used to enable or disable pH detector 510, conductivity detector 520 and contact detector 530 as well as other sensors (not shown) when it clear from the temperature of the oral appliance that the device is not in the patient's oral cavity. As further illustrated in FIG. 5, pH detector 510 provides an information signal via connection 515. Conductivity detector 520 provides an information signal via connection 525. Contact detector 530 provides an information signal via connection 535.

As described above, one or more conditions indicative of the presence or non-presence of the oral appliance in an oral cavity can be communicated as a binary condition after a comparison of a measured value with a predetermined threshold. Alternatively, one or more of the sensors in sensor assembly 330 may forward a measured value for comparison with one or more thresholds stored in controller 352. Although sensor assembly 330 is depicted as a multiple sensor device, it should be understood that for some hosts it may be desired to arrange the sensor assembly 330 with a single sensor.

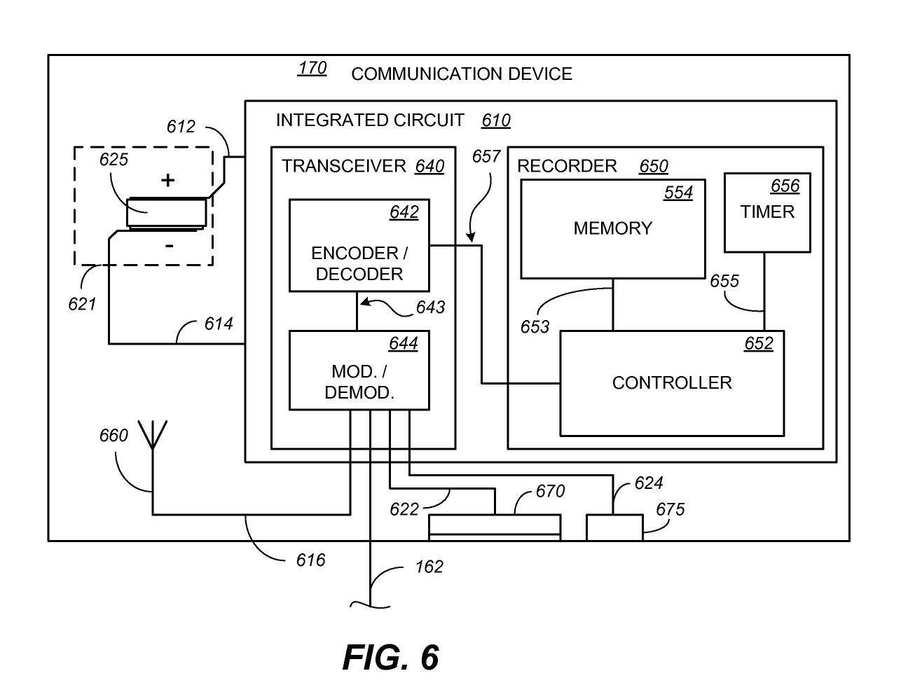

FIG. 6 is a functional block diagram illustrating an embodiment of the communication device 170 of FIG. 1. Communication device 170 includes integrated circuit 610, power source 621, antenna 660, photosensor (infrared sensitive) 670 and light-emitting diode (LED) 675. Power source 621 provides power to integrated circuit 610. Specifically, a positive terminal of power source 621 is electrically connected to a power input pin via conductor 612 and a negative terminal of power source 621 is electrically connected to a ground input pin via conductor 614. In the illustrated embodiment, power source 621 includes battery 625. When communication device 170 is coupled to data analyzer 160 (FIG. 1) conductors within wired link 162 may be dedicated to providing various voltages to integrated circuit 610 in lieu of power supply 621. In addition, wired link 162 may be coupled to a circuit (not shown) configured to charge a rechargeable battery 625 in power supply 621.

Integrated circuit 610 includes recorder 650 and transceiver 640. Recorder 650 is coupled to transceiver 640 via link 657. Transceiver 640 is coupled to antenna 660 via link 616. Transceiver 640 is also coupled to wired link 162 as well as photosensor 670 and LED 675. Photosensor 670 is coupled to transceiver 640 via link 622. LED 675 is coupled to transceiver 640 via link 624. Photosensor 670 detects incident light in the infrared band of frequencies. LED 675 emits light in the infrared band of frequencies in accordance with a modulated signal provided by transceiver 640. LED 675 and photosensor 670 enable line of sight communication via IrDA or other communication protocols that use infrared frequencies to communicate information wirelessly.

Recorder 650 includes controller 652, memory 654 and timer 656. Controller 652 is coupled to memory 654 via bus 653. Controller 652 is further coupled to timer 656 via link 655. Transceiver 640 includes encoder/decoder 642 and modulator/demodulator 644. Encoder/decoder 642 is coupled to modulator/demodulator 644 via bus 643.

In operation, an incident wireless signal containing information is received via antenna 660 and a tuner (not shown) or via photosensor 670. When received via antenna 660, the received radio-frequency signal is forwarded to modulator/demodulator 644 via link 616. When received via photosensor 670, the received signal is forwarded to modulator/demodulator 644 via link 622. When communication device 170 is coupled via a wired connection to a data analyzer 160 (not shown), commands can be forwarded to modulator/demodulator 644 via link 162. Modulator/demodulator 644 detects and separates information from the received signal. The information is forwarded to encoder/decoder 642 via bus 643. Encoder/decoder 642 converts the received information to a format compatible with controller 652. Controller 652 responds in accordance with the one or more received commands. For example, when enabled, communication device 170 is configured to send an identifier request command. When a suitably configured oral appliance 120 is within range and responds with its unique identifier, communication device 170 forwards a command(s) directing the oral appliance 120 to apply a select operational mode. The oral appliance 120 records information throughout the entire time that the oral appliance 120 is being used by the patient. When the oral appliance 120 is physically returned to a location where communication device 170 is present, oral appliance 120 receives a command from communication device 170 that instructs oral appliance 120 to communicate recorded information to communication device 170. In turn, communication device 170 will buffer the recorded information until it can be further communicated to data analyzer 160 (not shown). Data transfers between communication device 170 and data analyzer 160 may be interlaced with data being received from oral appliance 120.

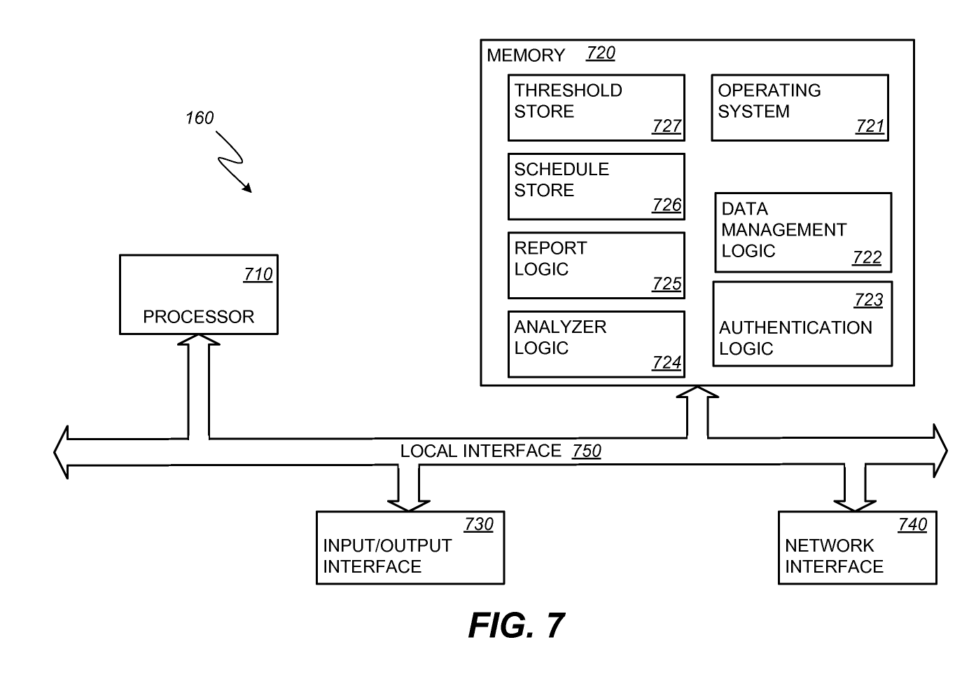

FIG. 7 is a functional block diagram illustrating an embodiment of the data analyzer 160 of FIG. 1. When the logic used by data analyzer 160 is implemented in software, as is shown in FIG. 3, it should be noted that one or more of data management logic 722, authentication logic 723, analyzer logic 724 and report logic 725, as well as information in schedule store 726 and threshold store 727 may be stored on any computer-readable medium for use by or in connection with any computer-related system or method. In the context of this document, a computer-readable medium is an electronic, magnetic, optical, or other physical device or means that can contain or store a computer program and data for use by or in connection with a computer-related system or method. The various logic elements and data stores may be embodied in any computer-readable medium for use by or in connection with an instruction execution system, apparatus, or device, such as a computer-based system, processor-containing system, or other system that can fetch the instructions from the instruction execution system, apparatus, or device and execute the instructions. In the context of this document, a “computer-readable medium” can be any means that can store, communicate, propagate, or transport the program for use by or in connection with the instruction execution system, apparatus, or device.

The computer-readable medium can be, for example but not limited to, an electronic, magnetic, optical, electromagnetic, infrared, or semiconductor system, apparatus, device, or propagation medium. More specific examples (a non-exhaustive list) of the computer-readable medium would include the following: an electrical connection (electronic) having one or more wires, a portable computer diskette (magnetic), a random access memory (RAM) (electronic), a read-only memory (ROM) (electronic), an erasable programmable read-only memory (EPROM, EEPROM, or Flash memory) (electronic), an optical fiber (optical), and a portable compact disc read-only memory (CDROM) (optical). Note that the computer-readable medium could even be paper or another suitable medium upon which the program is printed, as the program can be electronically captured, for instance via optical scanning of the paper or other medium, then compiled, interpreted or otherwise processed in a suitable manner if necessary, and then stored in a computer memory.

In an alternative embodiment, where data analyzer 160 is implemented in hardware, it may be implemented with any or a combination of the following technologies, which are each well known in the art: a discrete logic circuit(s) having logic gates for implementing logic functions upon data signals, an application specific integrated circuit (ASIC) having appropriate combinational logic gates, a programmable gate array(s) (PGA), a field programmable gate array (FPGA), etc.

In terms of hardware architecture, as shown in FIG. 7, data analyzer 160 includes a processor 710, memory 720, input and/or output (l/O) interface(s) 730, and network interface 740 that are in communication with each other via local interface 750. Local interface 750 can be, for example but not limited to, one or more buses or other wired or wireless connections, as is known in the art. The local interface 750 may have additional elements, which are omitted for simplicity, such as controllers, buffers (caches), drivers, repeaters, and receivers, to enable communications. Further, the local interface may include address, control, and/or data connections to enable appropriate communications among the aforementioned components.

The processor 710 is a hardware device for executing software, particularly software stored in memory 720. The processor 710 can be any custom made or commercially available processor, a central processing unit (CPU), an auxiliary processor among several processors associated with data analyzer 160, a semiconductor based microprocessor (in the form of a microchip or chip set), or generally any device for executing software instructions.

Memory 720 may include any one or combination of volatile memory elements (e.g., random access memory (RAM, such as DRAM, SRAM, SDRAM, etc.)) and nonvolatile memory elements (e.g., ROM, hard drive, tape, CDROM, etc.). Moreover, the memory 720 may incorporate electronic, magnetic, optical, and/or other types of storage media. Note that the memory 720 can have a distributed architecture, where various components are situated remote from one another, but can be accessed by the processor 710.

The software in memory 720 may include one or more separate programs, each of which comprises an ordered listing of executable instructions for implementing logical functions. In the example of FIG. 7, the software in the memory 720 includes data management logic 722, authentication logic 723, analyzer logic 724 and report logic 725 as well as information in schedule store 726 and threshold store 727. Memory 720 includes a suitable operating system (O/S) 721 and perhaps other application(s) or programs. The operating system 721 essentially controls the execution of other computer programs and provides scheduling, input-output control, file and data management, memory management, and communication control and related services.

Data management logic 722 is a database product that organizes and holds patient specific information received from each identified oral appliance such that the information can be accessed and retrieved in a number of different ways. When data analyzer 160 is hosted on a network coupled server, data management logic 722 will present one or more graphical user interfaces to enable a professional such as a an orthodontist, a dentist, a medical doctor or those under the control and supervision of the professional to manage patient accounts. Management of patient accounts includes adding, manipulating or removing patient specific information, associating data received from a patient's oral appliance 120 with the appropriate patient, setting alarms or warning message triggering conditions, etc.

Authentication logic 723 presents a user interface to each operator of a computing device that attempts to access information, including patient specific information, schedules, thresholds, etc. within data analyzer 160. The user interface directs the operator to enter appropriate credentials to authenticate the operator of the computing device communicating with the data analyzer 160. Upon receipt of a valid username and passcode, authentication logic 723 exposes appropriate data to the operator. For example, an administrator of the system is granted access to all interfaces, information, schedules, etc. A parent or other caregiver of a minor child is granted access to report interfaces and information related only to those in their care that are patients. A patient may be granted access to more or less information and reports in accordance with system parameters that may be applied by the system administrator or the parent/caregiver. Additional information that may be provided only to a patient may include motivational messages or suggestions from the orthodontist or other patients. An orthodontist is granted access to data to all patients under their care using appropriately configured oral appliances.

Analyzer logic 724 performs calculations to determine whether the information received from the oral appliance 120 indicates that the device was being worn. Analyzer logic 724 generates a result in response to information received from one or more of the sensors within oral appliance 120. Analyzer logic 724 uses the information to determine over what measurement intervals the oral appliance 120 was actually in the patient's oral cavity 115. In this regard, measurement information from adjacent data collection times consistent with the environment in an oral cavity defines a use interval. When a subsequent measurement indicates that the oral appliance is no longer in the oral cavity, the patient is credited for use up to last recorded use interval and a non-use interval is defined. In this way, data analyzer 724 generates a series of intervals and identifies each as a period of use or non-use for reporting purposes.

Report logic 725 includes instructions to generate various graphical-user interfaces and notices in response to use and non-use intervals, statistics and other information provided by analyzer logic 724. In this regard, report logic 725 may include a web server, an email generator, and an automated voice messaging system. Each of the web server, email generator and automated voice messaging system will be used under different circumstances. For example, the web server will collect, organize and forward information that can be used by a suitably configured computing device with a web browser, such that an operator of the computing device can observe the information. The email generator communicates notices to caregivers and/or patients at certain times or upon the occurrence of certain conditions under the direction and control of a system administrator. Similarly, the automated voice messaging system communicates messages to caregivers and/or patients at certain times under the direction and control of a system administrator. The notices and messages communicated can be generated, modified and stored by the orthodontist or operators associated with the orthodontist as may be desired. For example, an orthodontist may request that all notices and messages sent to a patient be recorded in a patient specific log.

Schedule store 726 holds or buffers information that defines a minimum therapeutic use of oral appliance 120. A schedule stored in schedule store 726 is under the control and direction of the orthodontist. The schedule can be modified on a patient by patient basis to account for any unique patient conditions. Threshold store 727 holds or buffers one or more use minimums based on periodic intervals. For example, threshold store 727 may include a daily use threshold, a weekly use threshold and/or a monthly use threshold.

It will be appreciated that functional portions of data analyzer 160 may be a source program, executable program (object code), script, or any other entity comprising a set of instructions to be performed. When a source program, the program needs to be translated via a compiler, assembler, interpreter, or the like, which may or may not be included within the memory 720, so as to operate properly in connection with operating system 721. Furthermore, portions of data analyzer 160 may be written in (a) an object oriented programming language, which has classes of data and methods, or (b) a procedure programming language, which has routines, subroutines, and/or functions.

I/O interface(s) 730 may include circuits and buffers for coupling input devices, for example but not limited to, a keyboard, mouse, scanner, microphone, etc. to local interface 750. I/O interface(s) 730 may also include circuits and buffers for coupling output devices, for example but not limited to, a printer, display, etc. to local interface 750.

Network interface 740 may comprise the various components used to transmit and/or receive data over a network. By way of example, the network interface 740 may include a device that can communicate both inputs and outputs, for instance, a modulator/demodulator (e.g., modem), wireless (e.g., radio frequency (RF)) transceiver, a telephonic interface, a bridge, a router, network card, etc.

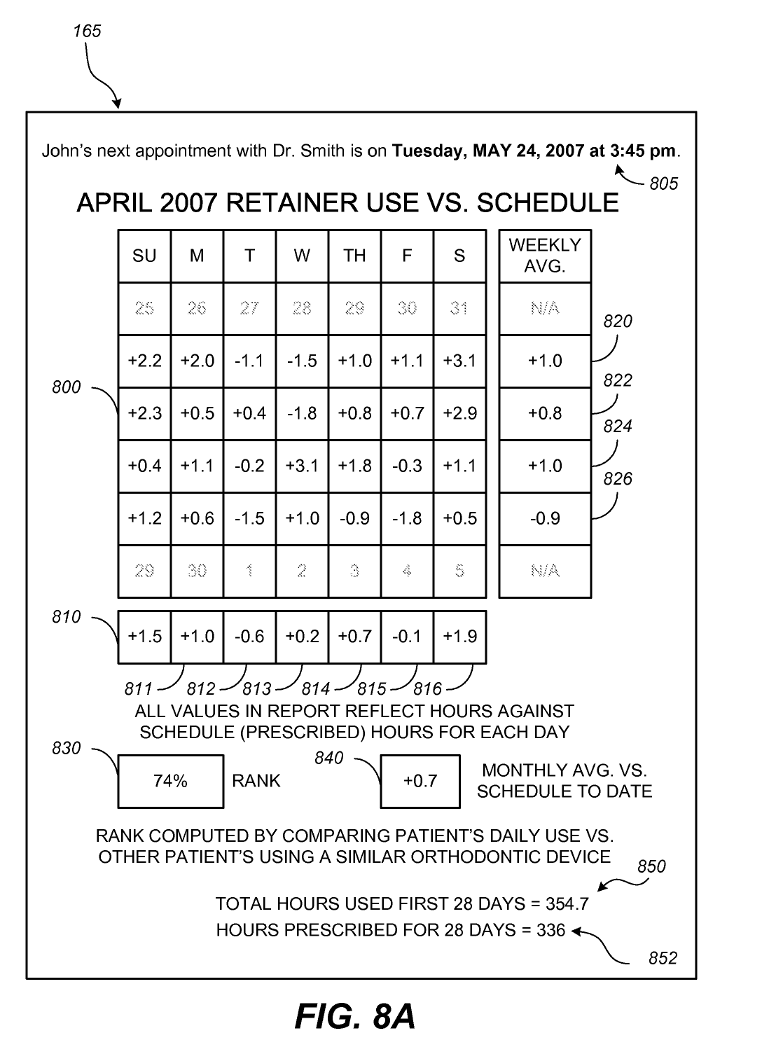

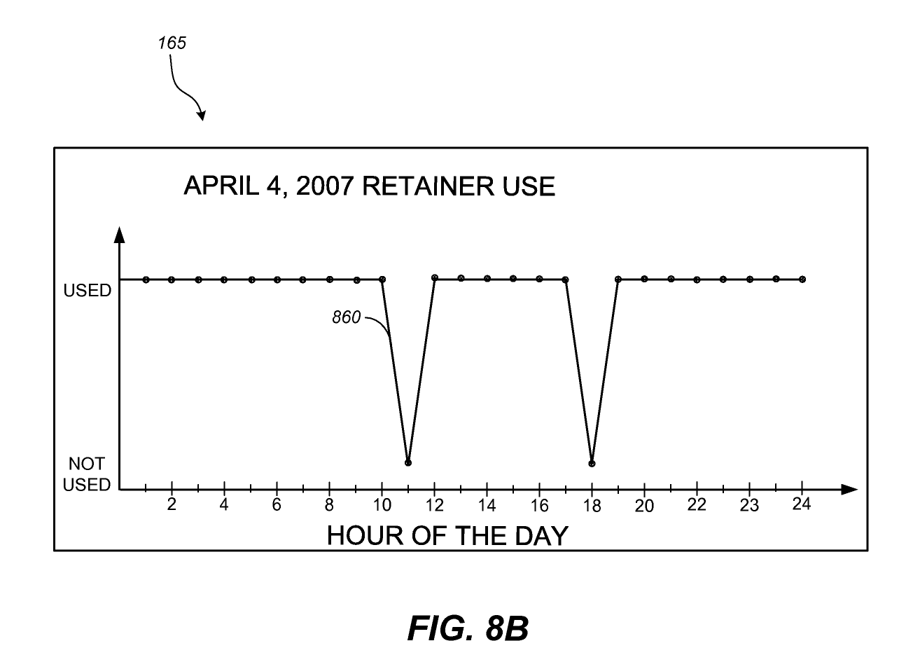

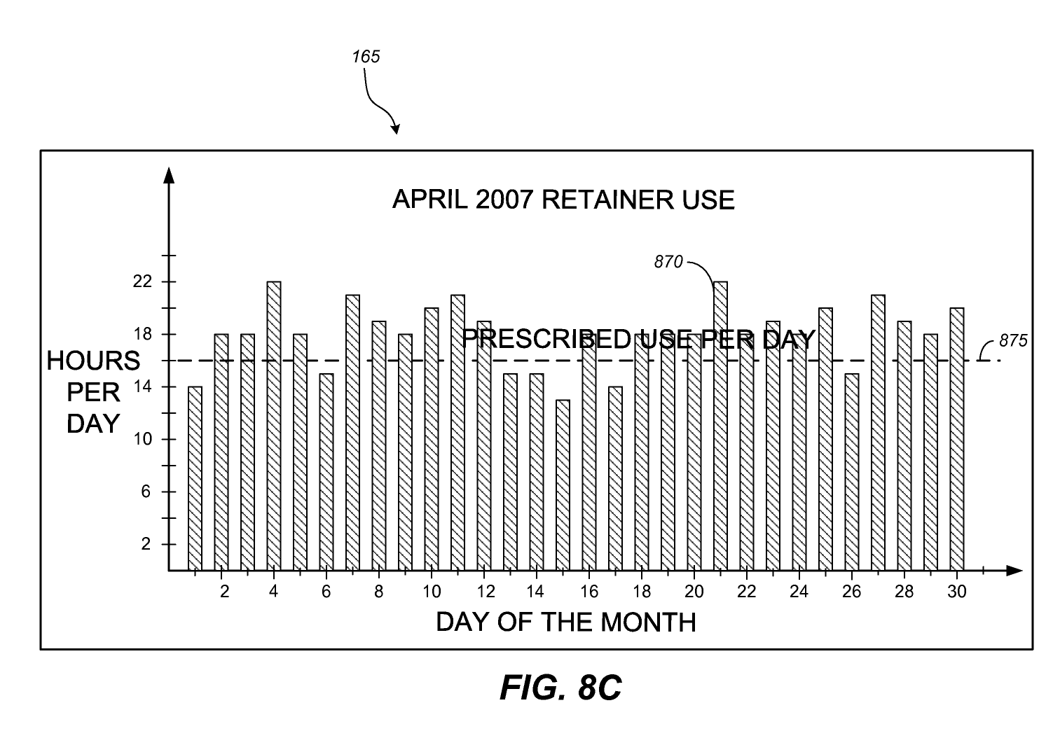

FIGS. 8A-8C are schematic diagrams illustrating alternative embodiments of a report generated by the data analyzer of FIG. 1. FIG. 8A illustrates an embodiment of a report 165 generated by the data analyzer 160 of FIG. 1. Report 165 includes a detailed report of retainer use with respect to a prescribed use for each day of the month of April in the year 2007. Report 165 includes a note 805 indicating when the patient is next scheduled for an office visit. Report 165 further includes array 800, which includes days in the month of April 2007 with a reported value in hours against a prescribed minimum schedule for use of the oral appliance 120 each day. As shown in array 800, when use exceeds the prescribed minimum a data value is positive. Conversely, when recorded use of the oral appliance 120 does not meet the minimum prescribed daily use a corresponding data value is negative. Report 165 further includes a weekly average use that is reported in corresponding fields. Field 820 presents the average use during the first week of April. Field 822 presents the average use during the second week of April. Field 824 presents the average use during the third week of April. Field 826 presents the average use during the fourth week of April.

Report 165 further includes a day-of-the-week average use that is reported in corresponding fields. Field 810 presents the average use during each Sunday during the month. Field 811 presents the average daily use each Monday during the month. Field 812 presents the average daily use each Tuesday during the month. Field 813 presents the average daily use each Wednesday of the month. Field 814 presents the average daily use each Thursday during the month. Field 815 presents the average daily use each Friday during the month. Field 816 presents the average daily use each Saturday during the month. The day-of-the-week averages are useful for highlighting any regularly scheduled activities that may be affecting a patient's compliance with a prescribed schedule.

In addition, report 165 includes field 830, which presents a comparison of the patient's use during the month with other patients using the same oral appliance 120. Field 840 presents a monthly average to date. Note that in the example embodiment, data values have not been recorded for April 29 and April 30. Field 850 displays the total hours of use in the first 28 days of the month. Field 852 displays the total hours of prescribed use over the same period (i.e., 28 days).

It should be understood that report 165 represents only one embodiment of a presentation of information that can be generated by data analyzer 160. Additional formats can be generated. For example, portions of the report may present stored and comparative information, including statistical values other than an average and a percentile.

FIG. 8B illustrates an alternative report 165. Report 165 includes a plot with hour of the day shown along the horizontal or x-axis of the plot and used or not used shown along the vertical or y-axis of the plot. Report 165 includes information for use over the course of Apr. 4, 2007. As indicated by line 860, the retainer was used consistently between midnight and approximately 10:00 am. The retainer was used again between approximately midday and about 5:00 pm (approximately 17 on the y-axis) and again from about 7:00 pm (approximately 19 on the y-axis) and through to midnight.

FIG. 8C illustrates a third embodiment of report 165. Report 165 includes a monthly use bar chart with day of the month presented along the horizontal or x-axis and hours of use per day shown along the vertical or y-axis of chart. Each day of the month is represented by a vertical bar 870, the vertical height of which corresponds to the hours per day that the retainer was used by the patient. A prescribed use per day is indicated by horizontal line 875. Report 165 presents an easy to read view of the patient's compliance with the prescribed use over each day of the month of April in the year 2007.

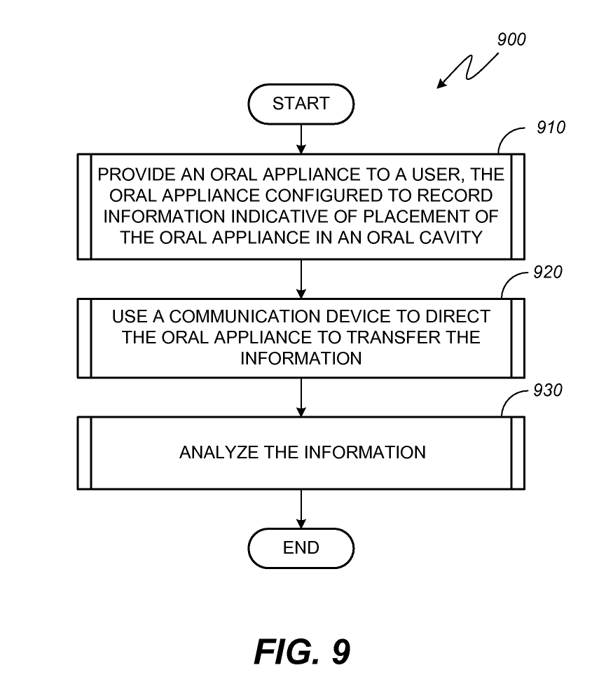

FIG. 9 is a flow diagram illustrating an embodiment of a method 900 for determining compliant use of an oral appliance 120. In this regard, the functions associated with blocks 920 and 930 represent respective specified steps or functions that can be embodied in software and/or a combination of hardware and firmware. When embodied in software and/or hardware/firmware, blocks 920 and 930 represent modules, segments, or portions of code, which comprise one or more executable instructions for implementing the specified function(s).

Method 900 begins with block 910 where an oral appliance 120 is provided to a user 110. As described above, the provided oral appliance 120 is configured to record a measurement or measurements indicative of placement in the oral cavity 115 of user 110.

In preferred embodiments, oral appliance 120 is directed to periodically record one or more specified measurements over a select length of time that does not exceed the capacity of a memory device to save the measurements. Thereafter, as indicated in block 920, the oral appliance 120 is directed to transfer the measurement or measurements. As shown in block 930, the received information is analyzed to see if it confirms the user's usage (i.e., placement) of the oral appliance 120 in the oral cavity 115.

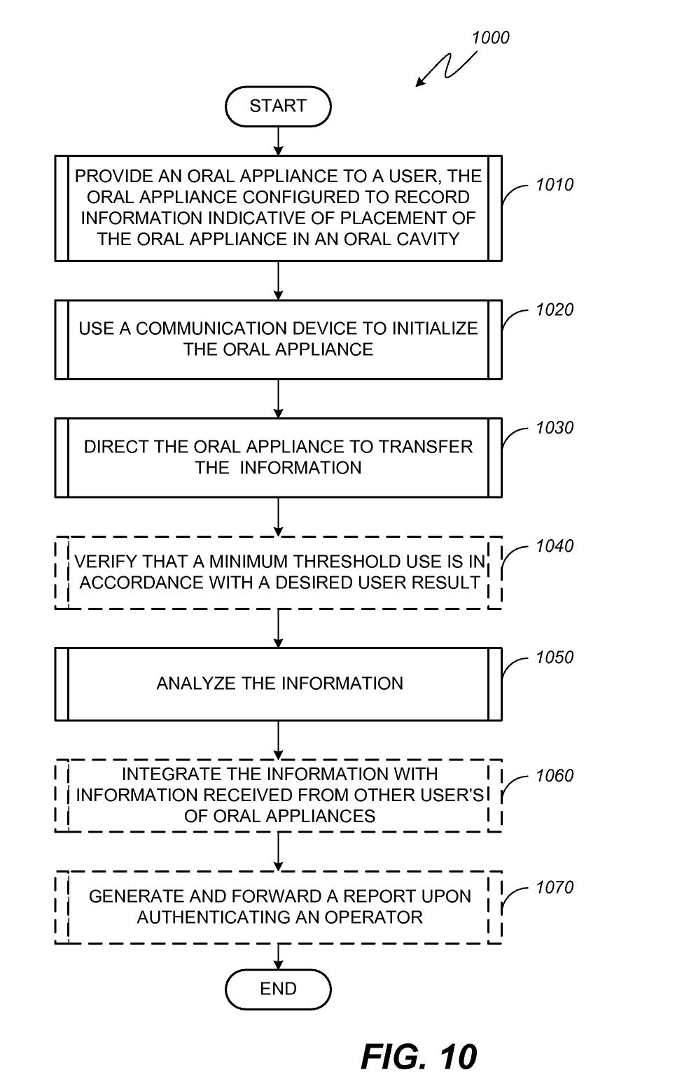

FIG. 10 is a flow diagram illustrating an alternative embodiment of a method 1000 for determining compliant use of an oral appliance 120. In this regard, the functions associated with blocks 1020, 1030, 1050, 1060 and 1070 represent respective specified steps or functions that can be embodied in software and/or a combination of hardware and firmware. When embodied in software and/or hardware/firmware, blocks 1020, 1030, 1050, 1060 and 1070 represent modules, segments, or portions of code, which comprise one or more executable instructions for implementing the specified function(s).

Method 1000 begins with block 1010 where an oral appliance 120 is provided to a user 110. As described above, the provided oral appliance 120 is configured to record a measurement or measurements indicative of placement in the oral cavity 115 of user 110. In preferred embodiments, oral appliance 120 is configured or initialized as indicated in block 1020 by a communication device. As described above, oral appliance 120 is configured to periodically record one or more specified measurements over a select length of time that does not exceed the capacity of a memory device to save the measurements. Thereafter, as indicated in block 1030, the oral appliance 120 is directed by a communication device to transfer the stored measurements. As shown in optional block 1040, a minimum threshold for use over a specified amount of total time is confirmed or verified that use in accordance with or that exceeds the minimum threshold will result in a desired outcome or result for the user of oral appliance 120. As indicated in block 1050, the received information is analyzed to see if it confirms the user's usage (i.e., placement) of the oral appliance 120 in the oral cavity 115 for the prescribed times. Thereafter, as indicated in optional block 1060 the received information is integrated with information received from other user's of oral appliances. Once the received information is integrated, as shown in optional block 1070, a data analyzer 160 or other computing device with access to the information, generates and forwards a report upon authenticating an operator of the computing device.

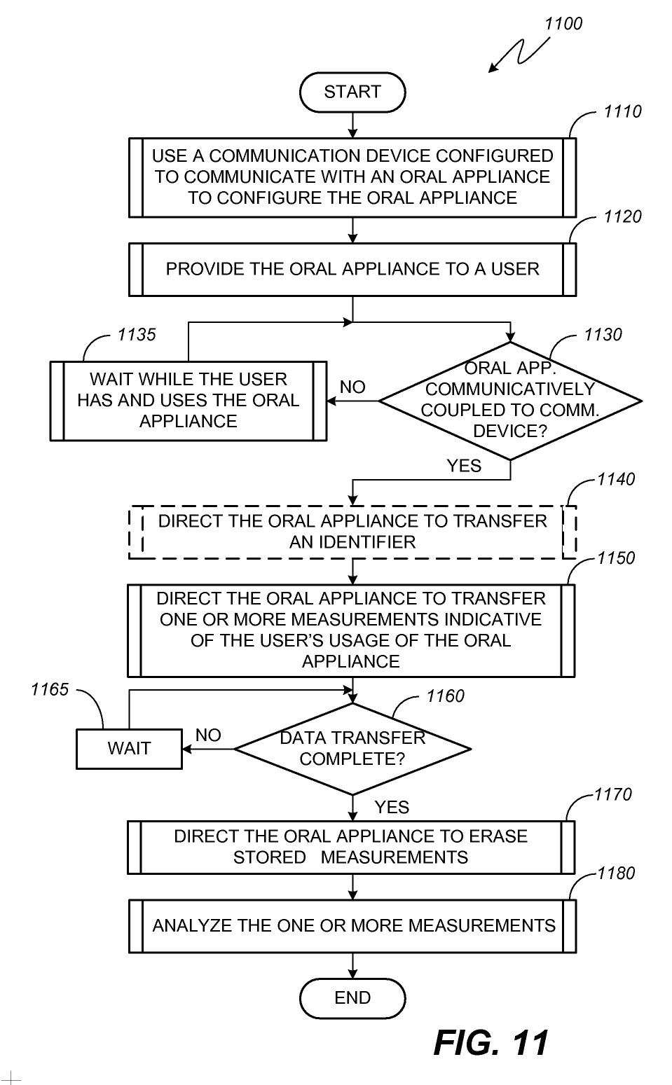

FIG. 11 is a flow diagram illustrating an embodiment of another method for determining compliant use of an oral appliance. In this regard, each block represents a specified step or function. When embodied in software and/or hardware/firmware, each block represents a module, segment, or portion of code, which comprises one or more executable instructions for implementing the specified function(s).

Operational software programs that may be used by a communication device 170, as well as operational software programs that may be used in conjunction with a data analyzer 160 communicatively coupled to the communication device 170, which comprise an ordered listing of executable instructions for implementing logical functions, can be embodied in any computer-readable medium for use by or in connection with an instruction execution system, apparatus, or device, such as a computer-based system, processor-containing system, or other system that can fetch the instructions from the instruction execution system, apparatus, or device and execute the instructions. Consequently, portions of method 1100 can be embodied on a computer-readable medium.

Alternative method 1100 begins with block 1110 where a communication device is used to communicate with and configure oral appliance 120. In block 1120, the configured oral appliance 120 is provided to a user. The user may be instructed at this or some other time to begin a scheduled or prescribed therapy by positioning oral appliance 120 in the user's oral cavity for a desired amount of time. In some embodiments, this prescribed or desired use may comprise a length of time for use of the device within each 24-hour period from receipt of the device until a future appointment. When the oral appliance 120 is configured to limit the volume of the oral cavity, as a weight loss aid, the oral appliance 120 may only need to be present during meal times to be effective. However, a physician or other party interested in the user's compliant use of oral appliance 120 may require the user to show use through a significant portion of each day. When this is the case, the oral appliance 120 may be configured to measure and record one or more environmental parameters in intervals shorter than 5 minutes. In other embodiments, such as when the oral appliance 120 is configured to apply pressure to a user's teeth, the oral appliance 120 may need to be present for a significant portion of each day to be effective. When this is the case, the oral appliance 120 may be configured to measure and record one or more environmental parameters in intervals of 5 minutes or more.

When the user 110 is in possession and is presumably using the oral appliance 120 in accordance with the prescribed or desired schedule, the oral appliance 120 is measuring and recording at least one environmental parameter every few minutes. The query of block 1130 and wait block 1135 are repeated until as indicated in query block 1130, oral appliance 120 is communicatively coupled to a suitably configured communication device. When this is the case, as indicated by the flow control arrow labeled, “YES,” exiting block 1130, method 1100 continues with optional block 1140, where the communication device 170 communicates a command to the oral appliance 120 to transfer an identifier. The identifier associated with the oral appliance 120 may be an alphanumeric string unique to the particular oral appliance 120 presently in close proximity (i.e., within operating range of the respective radio frequency or infrared transceivers or within the maximum distance permitted by a suitably configured cable) to the communication device 170. In alternative embodiments, the identifier may be a number or consist entirely of letters.

In block 1150, the communication device 170 directs the oral appliance 120 to transfer one or more measurements indicative of the user's use of the oral appliance 120 since it was first presented to the user and/or since the one or more measurements were transferred from the oral appliance 120. In block 1160, a query is performed to determine if the data transfer is complete. When the data transfer has not been completed, as indicated by the flow control arrow labeled, “NO,” exiting block 1160, a wait function or step is performed as indicated in block 1165. Thereafter, processing continues with the query of block 1160 until it is the case that the data transfer is complete. Once the data transfer is complete as indicated by the flow control arrow labeled, “YES,” exiting block 1160, the communication device 170 directs the oral appliance 120 to erase the stored measurements (in block 1170). Alternatively, communication device 170 directs the oral appliance 120 to use an operational mode that sets a pointer and records one or more data values from associated sensors in accordance with a select measurement interval. In block 1180, the communication device 170 or data analyzer 160 analyzes the one or more measurements to determine if the user's use of the oral appliance 120 is in accordance with one or more prescribed therapy schedules.

While various embodiments of the apparatuses, systems and methods for determining and reporting compliant use of an oral appliance have been described, it will be apparent to those of ordinary skill in the art that many more embodiments and implementations are possible that are within the scope of the accompanying claims. Accordingly, the apparatuses, systems and methods for determining and reporting compliant use of an oral appliance are not to be restricted beyond the attached claims and their equivalents.

What is claimed is:

1. An information system for determining compliant use of an oral appliance, comprising:
a communication device configured to communicate with the oral appliance when the oral appliance and communication device are proximal to one another, the oral appliance configured to periodically record at least one datum indicative of the presence of the oral appliance in an oral cavity; and
a data analyzer communicatively coupled to the communication device and configured to determine, from information originating in the oral appliance, whether a user's use of the oral appliance is compliant with a schedule.

2. The system of claim 1, wherein the oral appliance is prescribed by a professional.

3. The system of claim 1, wherein the oral appliance is removable.

4. The system of claim 1, wherein the oral appliance is a device configured to reduce the volume of a patient's oral cavity.

5. The system of claim 1, wherein the oral appliance is a device provided for the treatment of at least one of bruxism, sleep apnea and snoring.

6. The system of claim 1, wherein the communication device comprises a two-way transceiver for sending commands and receiving information from the oral appliance.

7. The system of claim 6, wherein the communication device directs one of a reset and an operational mode of the oral appliance, wherein the operational mode identifies at least one measure of an expected condition in a patient's oral cavity to record and an interval to wait before recording each subsequent data value.

8. The system of claim 6, wherein the communication device receives data selected from the group consisting of an identifier, a start time, a start date, an interval and data values.

9. The system of claim 1, wherein the data analyzer compares use of the oral appliance over one or more periods with a predetermined schedule.

10. The system of claim 1, wherein the data analyzer generates a report that is accessible to those with access privileges.

11. The system of claim 10, wherein the report is responsive to a comparison of use of the oral appliance with use of a group.

12. The system of claim 10, wherein the report comprises a relative ranking.

13. The system of claim 10, wherein the report comprises a message directed to the user of the oral appliance.

14. A method for confirming compliant use of an oral appliance, the method comprising:
providing an oral appliance to a user, the oral appliance configured to record information indicative of placement of the oral appliance in an oral cavity over time;
using a communication device to direct the oral appliance to transfer the information; and
analyzing the information to determine whether use of the oral appliance is compliant with a schedule.

15. The method of claim 14, wherein using a communication device comprises confirming the successful transfer of stored information to the communication device.

16. The method of claim 14, wherein using a communication device comprises directing the oral appliance to erase stored information.

17. The method of claim 14, wherein using a communication device comprises directing the oral appliance to overwrite stored information.

18. The method of claim 14, wherein using a communication device comprises directing the oral appliance to transfer an identifier.

19. The method of claim 14, wherein using a communication device comprises directing the oral appliance to encrypt data.

20. The method of claim 14, wherein analyzing the information comprises comparing indicia of two or more of time, temperature, humidity, contact and pH.

21. The method of claim 14, wherein analyzing the information comprises comparing use of the oral appliance with a predetermined schedule that defines a minimum threshold to obtain a desired therapeutic result.

22. The method of claim 21, further comprising:
verifying that the minimum threshold is in accordance with a desired user result; and
adjusting the schedule.

23. The method of claim 14, wherein analyzing the information comprises generating a report.

24. The method of claim 23, further comprising:
forwarding the report to a computing device upon validating one or more access credentials of an operator of the computing device.

25. The method of claim 23, wherein generating the report comprises providing a notice of the next scheduled patient visit with the orthodontist.

26. The method of claim 23, wherein generating the report comprises communicating a notice of non-compliant use to one or more of the orthodontist, patient and a patient's caregiver.

27. The method of claim 23, wherein generating the report comprises comparing use of the oral appliance with a group's use of respective oral appliances.

28. The method of claim 27, wherein generating the report comprises providing feedback via a relative ranking.

29. The method of claim 23, wherein generating the report comprises providing a total use over a select period.

30. The method of claim 14, further comprising:
integrating the information with information received from other oral appliances.
